SEQUENCE LISTING (7 sequences)

SEQ ID NO: 1 - protein, 20 aa, Artificial sequence
  Synthetic peptide
VRMNIQMLLEAADYLERRER

SEQ ID NO: 2 - protein, 14 aa, Artificial sequence
  Synthetic peptide
MNIQMLLEAADYLE

SEQ ID NO: 3 - protein, 14 aa, Artificial sequence
  Synthetic peptide
MNIQMLLEAPDYLE

SEQ ID NO: 4 - protein, 14 aa, Artificial sequence
  Synthetic peptide
MNIQMPLEAPDYLE

SEQ ID NO: 5 - protein, 11 aa, Artificial sequence
  Synthetic peptide
YGRKKRRQGGG

SEQ ID NO: 6 - RNA, 21 nt, Artificial sequence
  Synthetic oligonucleotide
gcaaagcggucccuguuuauu

SEQ ID NO: 7 - RNA, 21 nt, Artificial sequence
  Synthetic oligonucleotide
ggcaaugggucgugcgagauu

(19) United States
(12) Patent Application Publication
(10) Pub. No.: US 2014/0057961 A1
(43) Pub. Date: Feb. 27, 2014

(54) SIN3B COMPLEX INHIBITION AND USE THEREOF IN THE PREVENTION OF PRO-ONCOGENIC INFLAMMATION AND CANCER

(71) Applicant: New York University, New York, NY (US)

(72) Inventors: Gregory David, New York, NY (US); Maite Rielland, New York, NY (US)

(73) Assignee: New York University, New York, NY (US)

(21) Appl. No.: 13/973,075

(22) Filed: Aug. 22, 2013

Related U.S. Application Data
(60) Provisional application No. 61/692,914, filed on Aug. 24, 2012.

(51) Int. Cl. A61K 31/713

(52) U.S. Cl. CPC A61K 31/713
USPC 514/44 A

(57) ABSTRACT

Methods for inactivating Sin3B and its associated activities to prevent, inhibit or attenuate pro-oncogenic inflammation and cancer progression, in particular pancreatic cancer progression are provided.

This application claims the benefit of priority of U.S. Provisional Application No. 61/692,914, filed Aug. 24, 2012, the content of which is incorporated herein by reference in its entirety.

This invention was made with government support under contract numbers 5R21CA155736-02 and 5R01CA148639-03 awarded by the National Institutes of Health. The government has certain rights in the invention.

### INTRODUCTION

### Background

Pancreatic ductal adenocarcinoma (PDAC) carries a mean survival of only 6 months and is virtually always a fatal disease. The tumors are highly metastatic and show a striking resistance to existing therapeutic approaches. Non-invasive precursor lesions, known as pancreatic intraepithelial neoplasia (PanIN), are believed to represent initiating events in the generation of PDAC, as the progression from PanIN to PDAC has been recapitulated in mouse models (Hezel, et al. (2006) Genes Dev. 20:1218-1249). Studies involving genetically engineered mice and analysis of human specimens have demonstrated that KRAS mutations represent an early event in this process. They are found in the earliest PanINs that do not harbor any other known genetic alterations (Hezel, et al. (2006) supra). Surprisingly, PanINs have extremely low proliferation rates despite the fact that KRAS activation is an established driver in the proliferation of transformed cells. PanINs ultimately undergo stepwise progression, leading to increasingly dysplastic lesions, and culminating in PDAC, upon the acquisition of additional genetic alterations (Aguirre, et al. (2003) Genes Dev. 17:3112-3126; Habbe, et al. (2008) Proc. Natl. Acad. Sci. USA 105:18913-18918; Hingorani, et al. (2003) Cancer Cell 4:437-450; Klein, et al. (2002) Mod. Pathol. 15:441-447). The incidence of PanINs in the general population is estimated to be >30%, which is much higher than the lifetime risk of developing PDAC (Hruban, et al. (2001) Am. J. Surg. Pathol. 25:579-586). Together, these observations suggest that specific molecular mechanisms promote cellular proliferation during early stages of pancreatic cancer, and serve as potent pro-tumorigenic events. A series of genetically engineered mouse models faithfully recapitulates the histopathogenesis of human PDAC. These mice express a mutated allele of KRAS (KRAS^{G12D}) driven by its own promoter in pancreatic cells and develop PanIN lesions (Aguirre, et al. (2003) supra; Hingorani, et al. (2003) supra). Despite expression of the KRAS^{G12D} allele throughout the whole organ, only a few cells within the pancreas acquire a PanIN phenotype. A potential explanation of this focal development of premalignant lesions is that a defined threshold of KRAS signaling is required to initiate the neoplastic process, and that this occurs stochastically in a subset of cells. In line with this, PanIN lesions show much higher levels of activation of key pathways downstream of Ras, including MEK or ERK, compared to the adjacent normal tissues (Ji, et al. (2009) Gastroenterology 137:1072-1082). Thus, amplification of RAS signaling appears to be associated with PanIN development. While the expression of endogenous KRAS^{G12D} leads to the PanIN lesions in the mouse, the development of PDAC requires additional mutations. In particular, inactivation of p53 or of the INK4A/ARF locus results in the rapid progression of PanIN to PDAC, consistent with the presence of mutations in these genes in high grade PanIN and PDAC in human specimens (Aguirre, et al. (2003) supra; Bardeesy, et al. (2006) Proc. Natl. Acad. Sci. USA 103:5947-5952; Hingorani, et al. (2003) supra). Whereas these observations clearly establish a role for the ARF/p53 and the INK4a/Rb axes in limiting PDAC progression, the existing studies have not yet defined the specific molecular mechanisms preventing PanIN growth. This is due to the profound alterations in genomic stability, cellular survival, and other tumor suppressor functions regulated by these pathways (Sharpless (2005) Mutat. Res. 576:22-38; Sharpless & DePinho (2002) Cell 110:9-12).

Analyses have revealed an essential contribution of pro-inflammatory molecules in the initiation and progression of pancreatic cancer, both in these mouse models of PDAC and in human specimens. Specifically, the transcription factor Stat3, which is a well-established mediator of the inflammatory response, is essential for PDAC progression in mouse models of pancreatic cancer. Genetic inactivation of Stat3 in a mouse model of PDAC has indeed reveal the existence of a trans-signaling pathway, involving acinar cells-produced chemokines ands cytokines and the subsequent inflammatory response by myeloid cells, contributing to cancer progression (Corcoran, et al. (2011) Cancer Res. 71:5020-5029; Fukuda, et al. (2011) Cancer Cell 19:441-455; Lesina, et al. (2011) Cancer Cell 19:456-469). While additional factors, including NF-κB have been shown to contribute to this KRas-driven feed forward inflammatory loop (Li, et al. (2011) Cancer Cell 19:429-431; Ling, et al. (2012) Cancer Cell 21:105-120), identifying new targets could lead to the development of therapeutic approaches to target the disease at stages where treatment may be most effective.

The Sin3 complex is highly conserved throughout evolution, and mammalian cells contain two Sin3 proteins, Sin3A and Sin3B (Ayer, et al. (1995) Cell 80:767-776; Schreiber-Agus, et al. (1995) Cell 80:777-786; Vidal, et al. (1991) Mol. Cell. Biol. 11:6306-6316; Wang, et al. (1990) Mol. Cell. Biol. 10:5927-5936). Sin3 proteins are non-catalytic scaffold proteins that serve as an evolutionarily conserved component of the histone deacetylase HDAC1/2 transcriptional repression complex. A large and diverse group of sequence-specific transcription factors interact with the ubiquitously expressed Sin3A and/or Sin3B, and these interactions result in transcriptional repression (Silverstein & Ekwall (2005) Curr. Genet. 47:1-17). While most work has focused on its homolog Sin3A and its role as a co-repressor for numerous sequence-specific transcription factors (Silverstein & Ekwall (2005) supra), it has been recently demonstrated that the cellular functions of Sin3A and Sin3B are not redundant (Dannenberg, et al. (2005) Genes Dev. 19:1581-1595; David, et al. (2008) Proc. Natl. Acad. Sci. USA 105:4168-4172). Specifically, it was shown that both Sin3A and Sin3B are essential for embryonic development, but possess distinct properties (Dannenberg, et al. (2005) supra; David, et al. (2008) supra). Sin3A and the associated Sds3 protein are essential for the generation of repressed chromatin structure at pericentric loci, and their genetic inactivation results in aberrant chromosomal segregation and cell death (Dannenberg, et al. (2005) supra; David, et al. (2003) Genes Dev. 17:2396-2405). By contrast, Sin3B is dispensable for cellular viability (David, et al. (2008) supra). Studies have indicated that a Sin3B-containing complex interacts with Rb family tumor suppressor and regulates the transcriptional repression of E2F target genes upon cell cycle withdrawal (Balciunaite, et al. (2005) Mol. Cell. Biol. 25:8166-8178; David, et al. (2008) supra; Grandinetti & David (2008) Cell Cycle 7:1550-1554; Rayman, et al. (2002) Genes Dev. 16:933-947). Sin3B knockout mice and cells have been generated and it was demonstrated that Sin3B is essential for cell cycle exit upon quiescence and differentiation, through its ability to tether chromatin repressors on E2F target promoters (David, et al. (2008) supra; Grandinetti & David (2008) supra). These observations were expanded in the context of cellular senescence. Specifically, mouse embryonic fibroblasts genetically inactivated for Sin3B (Sin3B−/− MEFs) were, by contrast to their wild-type counterparts, refractory to replicative senescence and oncogenic Ras-induced senescence, as evidenced by continuous BrdU incorporation and decreased SA-β-gal staining (Grandinetti, et al. (2009) Cancer Res. 69:6430-6437). RasV12 expression leads to the direct recruitment of Sin3B and the Sin3B-dependent recruitment of chromatin repressors at pro-proliferative loci. Consistently, in the absence of Sin3B, E2F target genes fail to be transcriptionally repressed following RasV12 ectopic expression.

Purification of the complex(es) associated with Sin3 proteins identified the components of what has been defined as the core complex including HDAC1/2 (Alland, et al. (2002) Mol. Cell. Biol. 22:2743-2750; Doyon, et al. (2006) Mol. Cell. 21:51-64; Fleischer, et al. (2003) Mol. Cell.

Biol. 23:3456-3467; Kuzmichev, et al. (2002) Mol. Cell. Biol. 22:835-848; Lai, et al. (2001) Mol. Cell. Biol. 21:2918-2932; Skowyra, et al. (2001) J. Biol. Chem. 276:8734-8739). Additional unbiased proteomic approaches in mammalian systems have led to the purification of proteins that associate stably and specifically with Sin3B, but not Sin3A. These include Pf1 (also known as Phf12, likely to be the homolog of yeast Rco1), KDM5A (a histone H3 lysine 4 demethylase, also known as Jarid1A or Rbp2), MrgX, EMSY and GATAD1 (Bartke, et al. (2010) Cell 143:470-484; Hayakawa, et al. (2007) Genes Cells 12:811-826; Malovannaya, et al. (2011) Cell 145:787-799; Vermeulen, et al. (2010) Cell 142:967-980). Notably, these proteins were consistently found to associate with Sin3B in several independent purifications, suggesting they make up the full Sin3B-Pf1 complex. Pf1, EMSY and KDM5A are all significantly overexpressed in breast cancers (Beroukhim, et al. (2010) Nature 463:899-905) and Sin3B, in complex with Pf1 and HDAC1/2, modulate Polymerase II pausing at actively transcribed genes (Jelinic, et al. (2011) Mol. Cell. Biol. 31:54-62). Furthermore, the production of inflammatory cytokines was recently shown to be highly dependent on polymerase pausing (Adelman, et al. (2009) Proc. Natl. Acad. Sci. USA 106:18207-18212; Gilchrist, et al. (2012) Genes Dev. 26:933-944; Hargreaves, et al. (2009) Cell 138:129-145).

### SUMMARY OF THE INVENTION

This invention is a method for preventing, attenuating or inhibiting pro-oncogenic inflammation by administering to a subject in need thereof an effective amount of an inhibitor of a protein of the Sin3B Complex. This invention is also a method for preventing, attenuating or inhibiting initiation or progression of cancer by administering to a subject in need thereof an effective amount of an inhibitor of a protein of the Sin3B Complex. In some embodiments, the subject of these methods has an activating mutation of a Kras oncogene, e.g., Kras^{G12D}. In other embodiments, the subject is suspected of having, or is at risk of having, pancreatic cancer. In certain embodiments, the protein of the Sin3B Complex is Sin3B, HDAC1, HDAC2, KDM5A, MeCP2, SMRT, Pf1, MrgX, EMSY or GATAD1. In particular embodiments, the inhibitor inhibits the expression of Sin3B or is a Sin3B scaffold inhibitor.

### BRIEF DESCRIPTION OF THE DRAWINGS

FIG. 1 shows that genetic inactivation of Sin3B delays progression of KRas^{G12D}-driven PanINs. Filled bars represent Sin3BP^{+/-}Kras^{pG12D} pancreas and open bars represent Sin3BP^{-/-}Kras^{pG12D} pancreas. Number of normal ducts (ND), metaplasic lesions (ML), PanIN1 (P1), PanIN2 (P2) and PanIN3 (P3) per field in 24 week old mice.

FIG. 2 shows that genetic inactivation of Sin3B increases survival in a model of pancreatic ductal adenocarcinoma. Kaplan Meier Survival curve of Sin3B^{p+/-}Kras^{pG12D} mice (solid line, n=42) and Sin3B^{p-/-}Kras^{pG12D} mice (dashed line, n=26) (p<0.05 at 300 days).

FIG. 3 shows that Sin3B deletion facilitates acinar regeneration after Caerulein-induced pancreatitis. Quantitative PCR for IL-1α and IL-6 mRNA expression in pancreas obtained from Sin3B^{p+/-} pancreas that are still exhibiting ADM lesions (Sin3B^{p+/-} ADM, n=3), in Sin3B^{p+/-} pancreas that appeared regenerated (Sin3B^{p+/-} Regenerated, n=2) and in Sin3B^{p-/-} pancreas (n=4). Sin3B^{p+/-} Regenerated and Sin3B^{p-/-} expressions are relative to the Sin3B^{p+/-} ADM expression. IL-1α (*p<0.00001), IL-6 (*p<0.05), NS=non significant.

FIG. 4 shows the Sin3B levels correlate with an inflammatory response. Quantitative PCR for Sin3B (*p<0.005) and IL-1α (*p<0.05) mRNA expression in BxPc3 pancreatic cancer cell lines. BxPc3 were infected with an empty vector or a shRNA against Sin3B (shSin3B). ShSin3B mRNA expressions are relative to the empty vector expression.

FIG. 5 shows the Sin3B levels correlate with an inflammatory response. Quantitative PCR for IL-1α and IL-6 mRNA expression in BxPc3 pancreatic cancer cell lines. BxPc3 has been treated 48 hours with vehicle DMSO, 0.2 μM (TSA 0.2) or 1 μM (TSA 1) of Trichostatin A. Sin3B and IL-1α mRNA expressions are relative to the expressions in DMSO (*p<0.05).

### DETAILED DESCRIPTION OF THE INVENTION

It has now been shown that genetic inactivation of the chromatin associated Sin3B corepressor impairs oncogenic KRas-driven PDAC initiation and progression. The results presented herein indicate that, in the absence of Sin3B, pancreatic cells are unable to produce the pro-inflammatory cytokines that promote cancer progression. In turn, circulating cells fail to be recruited within pancreatic regions surrounding early lesions, and fibrosis is decreased.

Therefore, the present invention provides a method for preventing, attenuating or inhibiting pro-oncogenic inflammation in a subject by inhibiting the expression or activity of a protein of the Sin3B Complex. Pro-oncogenic inflammation, as used herein, refers to inflammation that promotes cancer progression. In accordance with the method herein, a subject who is at risk for (e.g., by genetic predisposition or cancer recurrence), or is suspected of having (e.g., by exhibiting symptoms), pro-oncogenic inflammation is identified and administered an effective amount of an agent that inhibits the activity or expression of a protein of the Sin3B Complex. As used herein, the term “patient” or “subject” is an individual having symptoms of, or at risk for, pro-oncogenic inflammation. In certain embodiments, the subject has an activating mutation of the Kras oncogene. In one embodiment, the activating mutation of Kras is Kras^{G12D}. In another embodiment, the subject further has one or more mutations in the p53 or the INK4A/ARF locus. In certain embodiments, the subject has, or is at risk of having, pro-oncogenic inflammation associated with pancreatic cancer or another malignancy.

Patients may be human or non-human and may include, for example, animal strains or species used as “model systems” for research purposes, such a mouse model as described herein. A patient may include either adults or juveniles (e.g., children). The term “patient,” further refers to any living organism, preferably a mammal (e.g., human or non-human) that may benefit from the administration of compositions contemplated herein.

In specific embodiments, the method herein inhibits or reduces pro-oncogenic inflammation, by at least about 5%, 10%, 15%, 20%, 25%, 30%, 35%, 40%, 45%, 50%, 55%, 60%, 65%, 80%, 85%, 90%, 95%, or 100%, relative to inflammation observed prior to administration of an inhibitory agent described herein. In particular embodiments, the method for preventing, attenuating or inhibiting pro-oncogenic inflammation provided herein inhibits or reduces pro-oncogenic inflammation, in the range of about 5% to 10%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 99%, 30% to 100%, or any percentage in between, relative to inflammation observed prior to administration of an inhibitory agent described herein. The level of inflammation in a subject can be assessed by methods well known in the art, e.g., MRI, CT scan, or PET scan. Alternatively, the level of inflammation prior to and after administration of an inhibitory compound can be assessed by measuring levels of cytokines and/or chemokines including, but not limited to, IL-6, IL-1α, IL-1β, TNF-α and/or MMP7.

The prevention, attenuation or inhibition of pro-oncogenic inflammation in a subject finds application in inhibiting the initiation or progression of cancer, in particular a cancer mediated by an activating mutation of Kras, e.g., Kras^{G12D}. Prevention, attenuation or inhibition of cancer initiation or progression refers to preventing and/or slowing the formation and/or growth of a cancer disclosed herein, in particular, a cancer that may be localized or at an early stage, e.g., precancerous.

Activating mutations of KRas, alone or in combination with other mutations, are known in the art to promote various different types of cancers including, but not limited to, lung adenocarcinoma, pancreatic ductal adenocarcinoma, myeloproliferative disease, colon cancer, neurofibroma, and primary intrahepatic cholangiocarcinoma. Therefore, the instant method is particularly useful in preventing, attenuating or inhibiting the initiation or progression of lung adenocarcinoma, pancreatic ductal adenocarcinoma, myeloproliferative disease, colon cancer, neurofibroma or primary intrahepatic cholangiocarcinoma. In certain embodiments, an inhibitory agent described herein is used in the prevention, attenuation or inhibition of pancreatic cancer initiation or progression, in particular pancreatic ductal adenocarcinoma or precursor pancreatic intraepithelial neoplasis.

As is known in the art, “pancreatic cancer” refers to cancers that originate in the tissue of the pancreas, such as a pancreatic ductal adenocarcinoma cell. A “pancreatic ductal adenocarcinoma” refers to a cancer that originated from the ductal lining of the pancreas. A pancreatic ductal adenocarcinoma cell may be found within the pancreas forming a gland, or found within any organ as a metastasized cell or found within the blood stream of lymphatic system. “Pancreatic intraepithelial neoplasia” (PanIN) is a histologically well-defined precursor to invasive ductal adenocarcinoma of the pancreas.

Subjects who may receive benefit from the methods of this invention can be diagnosed using any suitable method, including but not limited to, biopsy, x-ray, or blood tests routinely performed in the art. For example, subjects having an activating mutation of Kras, e.g., Kras^{G12D}, can be identified by routine genetic testing.

Sin3A/B are large multidomain proteins that contain four paired amphipathic α-helices (PAH) known as PAH domains, a central HDAC interaction domain (HID) to which almost all of the core corepressor components bind, and a C-terminal highly conserved region (HCR). As well as serving as a bridge between transcription factors and histone deacetylation (HDAC) and histone demethylation activities (e.g., KDM5A or Jarid1A), the Sin3 Complex has also been shown to interact with the methylated DNA binding protein MeCP2 and the HDAC-associated corepressor silencing mediator of retinoic acid and thyroid hormone receptor (SMRT) (Silverstein & Ekwall (2005) Curr. Genet. 47:1-17), as well as Pf1, MrgX, EMSY and GATAD1 (Bartke, et al. (2010) supra; Hayakawa, et al. (2007) supra; Malovannaya, et al. (2011) supra; Vermeulen, et al. (2010) supra). Therefore, a “Sin3B Complex,” as used herein, is intended to include one or more of the following proteins: Sin3B, HDAC1, HDAC2, KDM5A, MeCP2, SMRT, Pf1, MrgX, EMSY and GATAD1. Any agent that inhibits the ability of one or more proteins to interact with Sin3B (referred to herein as a Sin3B scaffold inhibitor), or alternatively inhibits the associated enzymatic activities is considered a Sin3B Complex inhibitor that inhibits the activity of a protein of the Sin3B Complex.

Agents of use in the methods of the invention can inhibit either the expression or activity of a protein of the Sin3B Complex. In one embodiment, the Sin3B Complex inhibitor inhibits the activity of a protein of the Sin3B Complex. Agents of use in accordance with this embodiment of the invention typically include protein- or peptide-based inhibitors or small organic molecules. For the purposes of the present invention, a “protein-based” or “peptide-based” inhibitor is an inhibitor composed of two or more amino acid residues covalently attached by peptide bonds, which may be further modified to include organic and/or inorganic groups. Protein-based or peptide-based inhibitors include Sin3B binding proteins or peptides that bind to Sin3B thereby blocking the interaction between Sin3B and one or more chromatin modifying enzymes. One example of a suitable peptide-based inhibitor is a decoy peptide, e.g., a Sin3 interaction domain (SID) peptide, which interferes with recruitment of MAD and HDAC1 by Sin3B (Farias, et al. (2010) Proc. Natl. Acad. Sci. USA 107:11811-6; US 2011/0003753). SID decoy peptides of use in this invention include the peptides VRMNIQMLLEAADYLERRER (SEQ ID NO:1), MNIQMLLEAADYLE (SEQ ID NO:2), MNIQMLLEAPDYLE (SEQ ID NO:3), or MNIQMPLEAPDYLE (SEQ ID NO:4), which can optionally contain a leader sequence (YGRKKRRQGGG, SEQ ID NO:5) corresponding to the human immunodeficiency virus type 1 Tat arginine-rich RNA-binding motif (ARM), which has been mutated (RRR>GGG) to improve nuclear entry.

A variety of small organic molecules also find use in the methods of this invention. In particular embodiments, the small organic molecules inhibit the activity of enzymes associated with Sin3B. These molecules include histone deacetylation inhibitors such as romidepsin, hydroxamic acids (e.g., trichostatin A, vorinostat, belinostat, LAQ824 and panobinostat), cyclic tetrapeptides (e.g., trapoxin B), benzamides (e.g., entinostat, CI994 and mocetinostat), electrophilic ketones, and aliphatic compounds (e.g., phenylbutyrate and valproic acid); or histone demethylation inhibitors such as 8-hydroxyquinolines, flavanoids, catechols, and N-oxalyl glycine derivatives. See, King, et al. (2010) PLoS One 5:e15535; Lohse, et al. (2011) Bioorgan. Med. Chem. 19:3625-36.

In another embodiment, the Sin3B Complex inhibitor inhibits the expression of a protein of the Sin3B Complex. Agents of use in accordance with this embodiment of the invention are typically oligonucleotide-based inhibitors composed of two or more nucleotides (RNA or DNA) and/or peptide-nucleic acids that inhibit the expression and/or activity of Sin3B. Preferably, oligonucleotides-based inhibitors decrease the level of expression of an endogenous gene (e.g., by decreasing transcription of the Sin3B gene). In particular embodiments, oligonucleotides-based inhibitors include antisense oligonucleotides (ODNs), interfering RNA, ribozymes and DNAzymes as sequence-specific inhibitors of gene expression.

Antisense oligonucleotides can be complementary to the entire coding region of protein of interest, but more preferably is an oligonucleotide which is antisense to only a portion of the coding or noncoding region of a protein of interest, e.g., the Sin3B disclosed in GENBANK Accession No. NM_{—}015260. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense oligonucleotide can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid can be produced biologically using an expression vector into which a nucleic acid has been subcloned in an antisense orientation.

RNA-interfering inhibitors are small nucleic acid molecules that downregulate, inhibit, or reduce the expression of Sin3B. Non-limiting examples of such nucleic acid molecules include short interfering nucleic acid (“siNA”), short interfering RNA (“siRNA”), double stranded RNA (“dsRNA”), microRNA (“miRNA”), and short hairpin RNA (“shRNA”). Techniques for making these nucleic acid molecules are disclosed, for example, in U.S. Pat. Nos. 5,514,567; 5,561,222; 6,506,559; 7,022,828; 7,078,196; 7,176,304; 7,282,564; and 7,294,504; which are incorporated herein by reference in their entirety. Exemplary siRNA molecules include GCAAAGCGGUCCCUGUUUAUU (SEQ ID NO:6) and GGCAAUGGGUCGUGCGAGAUU (SEQ ID NO:7), which have been shown to inhibit Sin3B expression (van Oevelen, et al. (2008) Mol. Cell. 32:359-370).

The inhibitory agents described herein can also be administered in conjunction with other therapies. For example, in the case of cancer therapy, the agent may be administered in conjunction with conventional drug therapy and/or chemotherapy. In one embodiment, the agent is administered before chemotherapy.

The Sin3B Complex inhibitors described herein can be administered alone or in combination with a physiologically or pharmaceutically acceptable carrier, excipient, or stabilizer. The term “pharmaceutically acceptable” means a non-toxic material that does not interfere with the effectiveness of the biological activity of the active ingredients. The term “pharmaceutically-acceptable carrier” means one or more compatible solid or liquid fillers, diluents or encapsulating substances which are suitable for administration to a human or other vertebrate animal. The term “carrier” refers to an organic or inorganic ingredient, natural or synthetic, with which the active ingredient is combined to facilitate the application.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. In the preferred embodiment, administration is by injection. Typical formulations for injection include a carrier such as sterile saline or a phosphate buffered saline. Viscosity modifying agents and preservatives are also frequently added.

Optional pharmaceutically acceptable excipients especially for enteral, topical and mucosal administration, include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Binders are used to impart cohesive qualities to a solid dosage formulation, and thus ensure that a tablet or bead or granule remains intact after the formation of the dosage forms. Lubricants are used to facilitate tablet manufacture. Disintegrants are used to facilitate dosage form disintegration or “breakup” after administration, and generally include, but are not limited to, starch, sodium starch glycolate, alginine, gums or cross linked polymers, such as cross-linked PVP. Stabilizers are used to inhibit or retard decomposition reactions which include, by way of example, oxidative reactions. Surfactants may be anionic, cationic, amphoteric or nonionic surface active agents. If desired, the compositions may also contain minor amount of nontoxic auxiliary substances such as wetting or emulsifying agents, dyes, pH buffering agents, or preservatives.

Administration of an inhibitory agent of the invention can be by various routes including, but not limited to, intradermal, intramuscular, intraperitoneal, intravenous, subcutaneous, oral, and/or gastric routes. The outcome of the therapeutic and prophylactic methods disclosed herein is to at least produce in a subject a healthful benefit, which includes, but is not limited to, prolonging the lifespan of a subject, delaying the onset of one or more symptoms of cancer, in particular pancreatic cancer, and/or alleviating a symptom of cancer after onset.

The actual dosage amount of an inhibitory agent of this invention may be determined by physical and physiological factors such as age, sex, body weight, severity of the disease, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

Single or multiple doses of the inhibitory agent are contemplated. Desired time intervals for delivery of multiple doses can be determined by one of ordinary skill in the art employing no more than routine experimentation. As an example, subjects may be administered two doses daily at approximately 12 hour intervals. In some embodiments, the inhibitory agent is administered once a day.

The inhibitory agent may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc.

The invention will be further described in the following examples, which do not limit the scope of the invention described in the claims.

### Example 1

### Materials and Methods

Acinar Cell Isolation and Culture.

Primary acinar cell cultures were prepared by modifying published protocols (Sawey, et al. (2007) Proc. Natl. Acad. Sci. USA 104:19327-19332). Cultures were maintained on MATRIGEL using RPMI 1640 Medium (GIBCO) supplemented with 10% fetal bovine serum (FBS), penicillin G, streptomycin, 0.1 mg/ml Soybean Trypsin Inhibitor (Sigma-Aldrich), 1 μg/ml dexamethasone (Sigma-Aldrich), 2% MATRIGEL (BD Biosciences) and 1% glucose.

Animal Models.

The LSL-KRas^{G12D}, the p48-Cre, the LSL-Ink4a, and the mSin3B^{+/-} and mSinB^{L/L} strains have been described in the art (Jackson, et al. (2001) Genes Dev. 15:3243-3248; Kawaguchi, et al. (2002) Nat. Genet. 32:128-134; David, et al. (2008) Proc. Natl. Acad. Sci. USA 105:4168-4172; Aguirre, et al. (2003) Genes Dev. 17:3112-3126). The strains were mated to obtain mice with the correct genotypes. All animals were maintained in a mixed C57BL/6-FVB background.

Caerulein Treatment.

Acute caerulein pancreatitis was induced in 3- to 5-week-old mice with seven hourly intraperitoneal injections of Caerulein for three consecutive days (SIGMA) at a dose of 50 μg/kg. Control animals were injected with the same volume of phosphate-buffered saline (PBS). Pancreata were collected 1 hour and 48 hours after the final cerulein injection.

Cell Culture.

BxPc3 human pancreatic cancer cell lines are known in the art and available from American Type Culture Collection (ATCC, Manassas, Va.). Cells were cultured in RPMI, 10% FBS, Sodium Pyruvate, HEPES and Penicillin/Streptavidin. The cultures were maintained in 5% CO₂ at 37° C.

Gene Expression Microarray Analysis.

For microarray analysis, total RNA from mSin3B^{+/-}KRas^{G12D} and mSin3B^{-/-}KRas^{G12D} pancreas (two pancreata for each genotype) or PDEC (one for each genotype) were examined on AFFYMETRIX GENECHIP Mouse Genome 430A 2.0 Array. The data were analyzed using AGILENT GENESPRING GX11 (Agilent Technologies, Santa Clara, Calif.) to identify gene probes that showed more than a 1.5-fold change with statistical significance (P<0.05, unpaired t-test). The data were also analyzed using AFFYMETRIX EXPRESSION CONSOLE and Genepattern software for the calculation of average expression levels of each chromosome, with each array normalized with the robust multichip array (RMA) algorithm. Gene ontology analysis (GO) was then performed by uploading the microarray data to the Database for Annotation, Visualization and Integrated Discovery (DAVID).

Human Pancreatic Tissue Samples. A pilot tissue microarray (TMA) of 20 patients with pancreatic cancer and control patients was created. From each patient with pancreatic adenocarcinoma, cores of normal pancreas, pancreatitis, low grade PanIN lesion, high grade PanIN lesion, tumors from most cellular areas, tumors from most desmoplastic area, and metastatic tumors to the lymph nodes were submitted. As controls, patients with pancreas resection for neuroendocrine tumor, solid pseudo papillary neoplasm, serous cyst adenoma or metastatic tumors to the pancreas were used. The nature of the lesions was confirmed by a pathologist for each core. Each TMA was composed of 6 to 7 patients and 2 to 3 controls. The core diameter was 2 mm. Sections (5 μm) were cut from formalin-fixed paraffin-embedded samples for the purpose of immunohistochemistry. For orientation, a core of liver tissue was used.

Histology and Immunohistochemistry.

Mouse pancreata were fixed overnight in 10% formalin (Fisher) and processed for paraffin embedding. For histology, deparaffinized sections (5 μm) were stained with Gill's hematoxylin (Richard-Allan Scientific) and eosin Y (Protocol) followed by an alcohol dehydration series and mounting (PERMOUNT, Fisher). Trichrome staining was performed at the NYU School of Medicine Histopathology Core Facility. For Alcian Blue staining, deparaffinized sections (5 μm) were stained with Alcian blue solution, 30 minutes, at room temperature, counterstained with Gill's hematoxylin, and followed by an alcohol dehydration series and mounting (PERMOUNT, Fisher). For immunohistochemistry, deparaffinized sections (5 μm) were rehydrated and quenched in 1% hydrogen peroxide/methanol for 15 minutes, and antigen retrieval was performed in 10 mM sodium citrate and 0.1% TWEEN-20 (pH 6.0) for 15 minutes in a microwave oven. Blocking was done in 10% serum, 1% BSA, and 0.1% TWEEN-20 for 1 hour at room temperature, followed by incubation with the primary antibodies diluted in 1% BSA overnight at 4° C.

The following primary antibodies were used: rabbit anti-Sin3B (A-20; Santa Cruz), rabbit anti-a-Amylase (Sigma-Aldrich), rat anti-CK19 (TromaIII; Developmental Studies Hybridoma Bank), rabbit anti-phospho-Stat3 (Tyr705; D3A7; Cell Signaling), rabbit anti-HP1 gamma (phospho S83; Abcam), rat anti-mouse F4/80 (eBioscience); mouse anti-CD68 (KP1; Abcam); rat anti-mouse CD45 (BD Biosciences); mouse anti-NFκB, p65 active subunit, clone 12H11 (Millipore), rabbit anti-IL1 alpha (Abcam); rabbit anti-Dec1 (Adrian Harris, Oxford, UK), rabbit anti-phospho-p44/42 MAPK (Thr202/Tyr204; Cell Signaling), mouse anti-γH2AX (Ser 139; Upstate Cell Signaling Solutions). After incubating with secondary biotinylated antibodies and ABC solution (both from Vector Laboratories), sections were developed with 3,3′-diaminobenzidine tetrahydrochloride (Sigma-Aldrich). After counterstaining with Gill's hematoxylin (Sigma), slides were subjected to an alcohol dehydration series and mounted with PERMOUNT (Fisher). Slides were examined on a ZEISS AXIOIMAGER A2 microscope.

Infection of Pancreatic Cancer Cells.

DNA sequences encoding shRNAs for Sin3B (shSin3B) were selected and cloned into the PLKO retroviral vector (Open Biosystem). BxPc3 cells were infected with shSin3B or PLKO for 3 days and selected in puromycin for 5 days. The level of Sin3B knockdown by shSin3B was determined by quantitative RT-PCR.

Immunoblot Analysis.

Cells were lysed in 1× RIPA buffer (1% NP-40, 0.1% SDS, 50 mM Tris-HCl pH 7.4, 150 mM NaCl, 0.5% Sodium Deoxycholate, 1 mM EDTA), 0.5 μM DTT, 25 mM NaF, 1 mM Sodium vanadate, 1 mM phenylmethanesulfonyl fluoride (PMSF) and protease inhibitors. The following primary antibodies were used: rabbit anti-phospho-p44/42 MAPK (Thr202/Tyr204; Cell Signaling), mouse anti-ERK (Cell Signaling), rabbit antiphospho-Stat3 (Tyr705; D3A7; Cell Signaling), rabbit anti-Stat3 (Cell Signaling). After incubation with either the secondary IRDye ALEXA Fluor 680 goat anti-mouse antibody or 800 goat anti-rabbit antibody (ODYSSEY), the membranes were visualized with the ODYSSEY Infrared Imaging System (Li-Cor).

Immunofluorescence.

Pancreata were removed, fixed in 4% paraformaldehyde overnight, washed in a 10% sucrose solution and snap-frozen in OCT compound (Tissue-Tek). Frozen sections of 5 μm were air-dried, permeabilized with 0.2% TRITON X-100 for 20 minutes, and blocked with 10% serum/0.1% TWEEN-20 for 1 hour. Slides were incubated with primary antibodies diluted in 1% BSA/0.1% TWEEN-20 overnight at 4° C. Slides were then incubated with ALEXA Fluor-labeled secondary antibodies (Invitrogen) diluted in 1% BSA for 1 hour and mounted using VECTASHIELD mounting medium with DAPI (Vector Laboratories). Slides were examined on a ZEISS AXIOVERT 200M microscope. The following antibodies were used: rat anti-CK19 (TromaIII; Developmental Studies Hybridoma Bank) and rabbit anti-α-Amylase (Sigma-Aldrich). Slides were examined on ZEISS AXIOIMAGER A2. The 3D immunofluorescence on acinarto-ductal metaplasia spheres was performed using known methods (Lee & Bar-Sagi (2010) Cancer Cell 18:448-458). The spheres were counterstained with topoisomerase and the slides were analyzed with a ZEISS LSM510 microscope.

Isolation and Culture of PDEC.

Isolation and culture of PDEC were performed as previously described (Agbunag, et al. (2006) Methods Enzymol. 407:703-710; Lee & Bar-Sagi (2010) supra). PDEC were isolated from 5 week-old mice and propagated in MATRIGEL (BD Bioscience).

RT-PCR and Quantitative RT-PCR.

Extraction of total RNA from Pancreas and PDECs was performed using RNEASY mini kit (QIAGEN). For the pancreas, a piece was snap-frozen, ground, and the frozen powder was added to the RNEASY lysis buffer. Reverse transcription was done using Moloney murine leukemia virus polymerase and oligo(dT) primers. RT-PCR analyses were done using Tag DNA polymerase (5 Prime) and dNTP (Promega). Quantitative RT-PCR analyses were done using the SYBR Green method and the samples were run on the BIO-RAD I cycler MYIQ. Expression levels were normalized to GAPDH. Results were reported as relative to the abundance of mSin3B^{+/-} or mSin3^{B+/-} KRas^{G12D} transcripts.

Senescence-Associated-β-Galactosidase (SA-β-gal) Assay.

Frozen sections of pancreatic tissue were fixed with 2% formaldehyde/0.2% glutaraldehyde in PBS for 3-5 minutes, washed with PBS, stained at 37° C. for 12-16 hours in XGal solution (1 mg/ml X-Gal, 5 mM potassium ferrocyanide, 5 mM potassium ferricyanide, and 1 mM MgCl₂ in PBS at pH 6.0). After counterstaining with Eosin (Richard-Allan Scientific), slides were subjected to an alcohol dehydration series and mounted with PERMOUNT (Fisher) and counterstained with Eosin (Protocols). Slides were examined on ZEISS AXIOIMAGER A2.

Statistical Analyses.

Data were analyzed by Student's t-test (paired, two-tailed) and results were considered significant at p-value <0.05. Results are presented as mean and standard error (±SE). Survival curves were plotted by the Kaplan-Meier method and compared by the log-rank test. The correlative relationships between two quantitative measurements were investigated using Spearman rank-order correlation and the chi-square statistic.

### Example 2

### Genetic Inactivation of Sin3B Delays Progression of KRas^{G121}-Driven PanINs

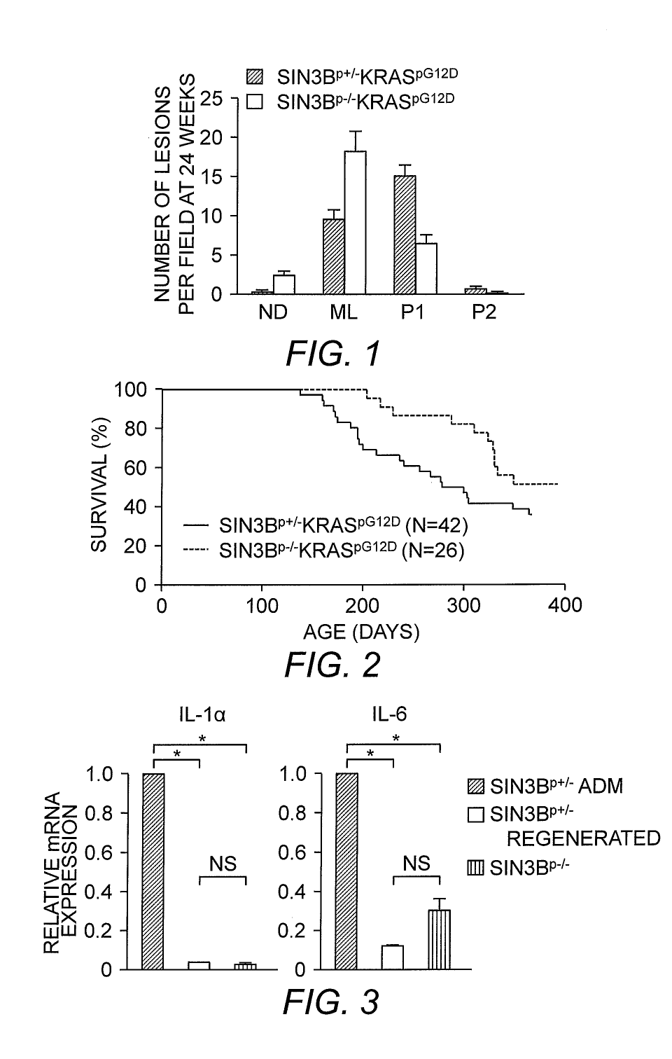

To examine the potential significance of Sin3B upregulation in PanIN lesions (Grandinetti, et al. (2009) Cancer Res. 69:6430-37), the impact of Sin3B deletion in the pancreas was analyzed. Mice carrying a Sin3B conditional allele were crossed with transgenic mice expressing the Cre recombinase under the control of the pancreas-specific p48 promoter (David, et al. (2008) Proc. Natl. Acad. Sci. USA 105:4168-4172; Kawaguchi, et al. (2002) Nat. Genet. 32:128-134). Sin3B^{flox/-}; p48-Cre⁺ animals (hereafter referred to as Sin3B^{p-/-}) were born at the expected ratio, showed no gross abnormalities at one year of age, and exhibited normal pancreatic morphology. Efficient Sin3B inactivation in Sin3B^{p-/-} pancreata was confirmed by transcript analysis and immunohistochemistry (IHC). Finally, the exocrine and endocrine functions of the pancreas appeared largely unaffected by the inactivation of Sin3B, as evidenced by the production of amylase and insulin in both Sin3B^{p+/-} and Sin3B^{p-/-} pancreata. Thus, Sin3B is largely dispensable for normal development of the pancreas. It was next investigated whether Sin3B deletion affects the progression of PanIN driven by pancreatic-specific activation of Kras^{G12D} (KRas^{pG12D}) by crossing the Sin3B^{p-/-} mice with the Cre-inducible Lox-STOP-Lox-KRas^{G12D} strain (Jackson, et al. (2001) supra). All genotypes (including Sin3B^{p+/-}KRas^{pG12D} and Sin3B^{p-/-}KRas^{pG12D} mice) were observed at the expected ratio, and efficient Sin3B deletion was confirmed. In mice sacrificed at six months of age, there were striking differences in the gross appearance of the pancreas. While the pancreas of Sin3B^{p+/-} KRas^{pG12D} mice was granular with abundant pale nodules throughout, signaling the presence of numerous metaplastic and PanIN lesions, the pancreas of age-matched Sin3B-deleted littermates (Sin3B^{p-/-}KRas^{pG12D}) exhibited normal gross appearance. Histologic examination of additional animals at serial time points revealed PanIN as early as 6-8 weeks and progressively higher grade lesions by 24 weeks in Sin3B^{p+/-}KRas^{pG12D} mice. By stark contrast, Sin3B^{p-/-}KRas^{pG12D} mice exhibited only rare early PanINs up to 24 weeks of age (n=11 for each genotype). These morphologic findings were corroborated by staining for CK19 and Alcian Blue, which together mark mucin-containing PanIN cells. Quantification of CK19-positive structures indicated a significant decrease in duct-like structures at 6-8 weeks and a delay in the progression of the pancreatic lesions at 24 weeks (FIG. 1) in the Sin3B^{p-/-}KRas^{pG12D} mice compared to their Sin3B^{p+/-}KRas^{pG12D} littermates. Importantly, morbidity was significantly delayed upon Sin3B deletion in KRas-expressing mice (FIG. 2; p<0.05 at 300 days). Thus, Sin3B deletion strongly impairs PanIN initiation and progression to advanced disease.

### Example 3

### Sin3B Deletion Impairs the Establishment but not The Initiation of Acinar-to-Ductal Metaplasia In Vivo

Recent lineage tracing studies have indicated that the majority of human and mouse PanIN lesions result from the transdifferentiation of acinar cells into ductal cells, through a process known as acinar-to-ductal metaplasia (ADM) (De La, et al. (2008) Proc. Natl. Acad. Sci. USA 105:18907-18912; Gidekel Friedlander, et al. (2009) Cancer Cell 16:379-389; Habbe, et al. (2008) Proc. Natl. Acad. Sci. USA 105:18913-18918). Histologic observations and the overall reduction of amylase staining strongly suggested that ADM had previously occurred in 8 week-old Sin3B^{p+/-} KRas^{pG12D} pancreata. By contrast, ADM was still observed in 8 week-old Sin3B^{p+/-}KRas^{pG12D} as evidenced by frequent co-expression of amylase and CK19. Together, these observations indicate that Sin3B deletion impaired ADM establishment but not the initiation of these lesions. To examine this process in further depth, acinar cells were isolated from 5 week-old Sin3B^{p+/-}KRas^{pG12D} mice or their Sin3B^{p+/-}KRas^{pG12D} littermates and subsequently cultured in a 3D matrix (Sawey, et al. (2007) supra). In cultures from both genotypes, amylase expression was detectable in acini upon initial isolation, and these cells progressively underwent ADM as evidenced by increased CK19 levels staining and formation of sphere like-structures by day 5. The efficiency of sphere formation was not significantly affected by Sin3B deletion. Together with the in vivo observations, these results indicate that, while Sin3B is dispensable for oncogene-driven ADM initiation, ADM establishment is affected by Sin3B deletion in vivo.

### Example 4

### Sin3B Deletion Facilitates Acinar Regeneration after Caerulein-Induced Pancreatitis

Upon KRas activation, ADM lesions arise and develop into PanINs (De La, et al. (2008) supra; Gidekel Friedlander, et al. (2009) supra; Habbe, et al. (2008) supra). ADM can also be initiated in response to injury such as pancreatitis, an inflammatory disorder that can be recapitulated in vivo upon caerulein treatment (Lerch & Gorelick (2013) Gastroenterology 144:1180-1193). Nevertheless, in absence of KRas, ADM is not sustained and the pancreas rapidly regenerates after injury (De La, et al. (2008) supra; Husain & Thrower (2009) Curr. Opin. Gastroenterol. 25:466-471). To assess the contribution of Sin3B in the maintenance of ADM lesions and pancreas regeneration, acute pancreatitis was induced by injecting caerulein for three consecutive days, hourly for seven hours in Sin3B^{p+/-} and Sin3B^{p-/-} mice. As expected, caerulein treatment promoted the formation of tubular complexes and metaplastic lesions in Sin3B^{p+/-} and Sin3B^{p-/-} mice with comparable efficiency when documented after 1 hour. Forty-eight hours after treatment, the pancreata of half of the Sin3B^{p+/-} experimental mice still exhibited ADM lesions, consistent with gradual pancreatic regeneration post treatment. By contrast, the pancreata of all Sin3B^{p-/-} mice appeared largely devoid of lesions and grossly normal. Both Sin3B^{p-/-} and Sin3B^{p+/-} pancreata that appeared regenerated 48 hours after the treatment (hereafter referred to as Sin3B^{p+/-} Regenerated) displayed a significant down-regulation of IL-1α and IL-6, two cytokines upregulated in pancreatitis (Yasuda, et al. (1999) J. Interferon Cytokine Res. 19:637-644), compared to Sin3B^{p+/-} pancreata exhibiting ADM lesions at that time (Sin3B^{p+/-} ADM) (FIG. 3). Of note, TNFα and MMP7 levels did not significantly differ between Sin3B^{p+/-} ADM and Sin3B^{p-/-}. Thus, Sin3B deletion facilitates acinar recovery after caerulein-induced pancreatitis and is associated with impaired IL-1α and IL-6 expression. Moreover IL-1α and IL-6 levels positively correlate with maintenance of ADM in vivo.

### Example 5

### Sin3B Deletion Impairs Oncogene Kras-Induced Senescence In Vivo

Consistent with a direct role of Sin3B in oncogenic Kras-induced senescence in vivo, Sin3B^{p-/-}KRas^{pG12D} pancreata displayed significantly reduced expression of established markers of senescence including Dec1, p15^{INK4B}, p21 and p53 transcripts as compared to those observed in Sin3B^{p+/-} KRas^{pG12D} littermates. Accordingly, staining for senescence-associated β-Galactosidase (SA-βgal) (Dimri, et al. (1995) Proc. Natl. Acad. Sci. USA 92:9363-9367) was markedly reduced in the Sin3B-deficient pancreata. These findings were exted by IHC analysis for phosphorylated HP1γ (P-HP1γ) and Dec1, which identify senescent cells in Sin3B^{p+/-}KRas^{G12D} PanINs (Caldwell, et al. (2012) Oncogene 31:1599-1608), but were undetectable in the rare PanINs arising in Sin3B^{p-/-} KRas^{pG12D} mice. Deletion of the Ink4a/Arf locus associated with the oncogenic KRas mutation in the pancreas leads to highly aggressive cancer and quickened mortality notably by bypassing senescence (Bardeesy, et al. (2006) Proc. Natl. Acad. Sci. USA 103:5947-5952; Lee & Bar-Sagi (2010) supra). To investigate if the delay in PanIN initiation and progression observed after Sin3B deletion is related to its function in the senescence process, Sin3B^{p+/-} KRas^{pG12D}Ink4a/Arf^{flox/flox} and Sin3B^{p-/-}KRas^{pG12D}Ink4a/Arf^{flox/flox} mice (hereafter referred to as Sin3B^{p+/-}KRas^{pG12D}Ink4a^{p-/-} and Sin3B^{p-/-}KRas^{pG12D}Ink4a^{p-/-}) (Aguirre, et al. (2003) supra; Bardeesy, et al. (02006) supra) were generated. Indeed, upon concomitant deletion of the Ink4a/Arf locus and KRas activation, Sin3B deletion did not confer a survival advantage. Both Sin3B^{p+/-}KRas^{pG12D}Ink4a^{p-/-} and Sin3B^{p-/-}KRas^{pG12D}Ink4a^{p-/-} mice exhibited advanced pancreatic cancer as evident by histology and diffuse CK19 expression. Absence of SA-βgal staining and p15^{INK4B} and Dec1 expression, and lack of IL-6 and IL-1α expression, two cytokines known to be upregulated in senescent cells as part of the SASP (Kuilman, et al. (2008) Cell 133:1019-1031), confirmed the bypass of senescence in the KRas^{pG12D}Ink4a^{p-/-} pancreas regardless of the Sin3B status. Thus, the diminished tumor progression caused by Sin3B deletion is paradoxically correlated with its ability to promote senescence, indicating an unexpected positive role of senescence in PDAC pathogenesis.

### Example 6

### Sin3B is Required for the Oncogenic KRas Pro-Inflammatory Response In Vivo

As it was found that Sin3B deletion is associated with impaired IL-6 and IL-1α in acute pancreatitis, and knowing that IL-6 and IL-1α are upstream regulators of the SASP (Orjalo, et al. (2009) Proc. Natl. Acad. Sci. USA 106:17031-36), it was subsequently determined whether Sin3B was involved in KRas-induced inflammation. The development of an inflammatory milieu composed of extensive immune cell infiltration, desmoplasia and cytokine secretion is fundamental to PanIN initiation and progression to PDAC (Bezel, et al. (2006) Genes Dev. 20:1218-49). Correspondingly, Sin3B^{p+/-}KRas^{pG12D} pancreata at age 6 weeks showed extensive Masson Trichrome staining which marks desmoplastic tissue. By contrast, Sin3B^{p-/-}KRas^{pG12D} pancreata only exhibited localized desmoplasia associated with rare PanINs at 24 weeks. Likewise, immune infiltration (CD45-, CD68- and F4/80-positive cells) was limited and highly localized in the Sin3B-deficient animals as compared to controls. Inflammation in evolving PanINs is associated with a positive feedback loop of cytokine secretion involving pancreatic cells, immune cells and cancer-associated fibroblasts (CAF) (Pylayeva-Gupta, et al. (2012) Cancer Cell 21:836-47; Tjomsland, et al. (2011) Neoplasia 13:664-75). The neoplastic cells mediate this process by secretion of inflammatory cytokines including IL-1α and IL-6, which are induced downstream of oncogenic KRas through activation of the ERK1/2, STAT3, and NF-kB pathways (Li, et al. (2011) Cancer Cell 19:429-431). Sin3B^{p+/-}KRas^{pG12D} PanINs presented strong nuclear signals for activated Stat3 (P-Stat3), ERK1/2 (P-ERK1/2) and p65 (active p65), whereas staining was virtually absent in the Sin3B-deleted PanINs. Western blot analysis of pancreata whole cell extracts confirmed the strong decrease in Stat3 and ERK1/2 activation upon Sin3B deletion. These effects were associated with strong reductions in the abundance of IL-6 and IL-1α transcripts in the Sin3B-deleted pancreas compared to their Sin3B-expressing littermates. Thus, the delayed progression of PanINs caused by Sin3B deletion is associated with a pronounced impairment in the inflammatory response.

### Example 7

### Sin3B is Required for Cell Autonomous IL-1 Alpha Expression

It was subsequently determined whether the defective induction of Kras-driven inflammation caused by Sin3B deletion involves cell autonomous mechanisms. To this end, global gene expression profiling was performed on Sin3B^{p+/-} KRas^{pG12D} and Sin3B^{p-/-}KRas^{pG12D} pancreata as well as corresponding cultured primary pancreatic duct epithelial cells (PDEC). Gene Ontology (GO) and pathway analysis using the David Functional Annotation tool revealed that the immune response was among the most significantly perturbed biological pathways in the Sin3B^{p-/-}KRas^{pG12D} pancreata compared to their Sin3B-expressing counterparts. In contrast to whole pancreata, few Sin3B-dependent changes were identified in primary PDEC cultures. For example, IL-6 was down-regulated in the Sin3B^{p-/-}KRas^{pG12D} pancreas, but not in Sin3B^{p-/-}KRas^{pG12D} PDEC cell lines. Similarly, the NF-κB and the IL-1R pathways were mostly down-regulated in Sin3B^{p-/-}KRas^{pG12D} pancreata compared to Sin3B^{p+/-}KRas^{pG12D} pancreata, but not in the PDEC cell lines. Importantly, IL-1α was among the few cytokines down-regulated both in the Sin3B^{p-/-} KRas^{pG12D} pancreas and PDEC lines. Further validating these observations, a significant reduction in IL-1α expression but not IL-6 was detected in two additional independent Sin3B^{p-/-}KRas^{pG12D} PDEC cell lines compared to their Sin3B^{p+/-}KRas^{pG12D} PDEC counterparts. These findings were extended in acinar cultures isolated from Sin3B^{p+/-}KRas^{pG12D} and Sin3B^{p-/-} KRas^{pG12D} mice, which revealed a specific reduction in IL-1α transcript levels in the Sin3B knockouts undergoing ADM. Thus, Sin3B plays a cell autonomous role in inducing IL-1α in KRas-expressing pancreatic cells.

### Example 8

### Sin3B Levels Correlates with an Inflammatory Response in Both Human Pancreatic Tissues and Cancer Cells

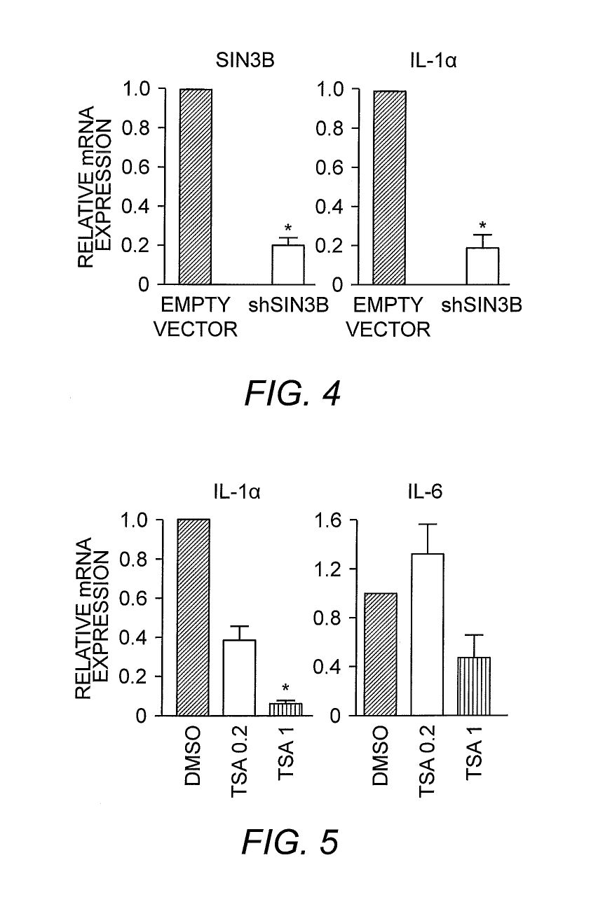

To investigate the relevance of Sin3B in human PDAC initiation and progression, Sin3B expression was examined using a human tissue array composed of 180 specimens including normal pancreas, pancreatitis, PanIN lesions, or PDAC. Sin3B was scarcely detected in control human pancreas or PDAC sections, but was strongly upregulated in both pancreatitis and PanINs, consistent with observations in mouse tissues (Grandinetti, et al. (2009) Cancer Res. 69:6430-6437). Interestingly, a positive correlation was observed between Sin3B, P-Stat3 and IL-1α positivity, specifically at the sites of ADM, and in PanINs in human tissues (correlation coefficient=0.2157; p=3.46E-12; n=156). Having uncovered the correlation between Sin3B expression level and inflammatory factors expression level in human pancreatic tissue, it was posited that Sin3B may modulate IL-1α expression in human pancreatic cells. Expression of shRNA targeting Sin3B (shSin3B) in BxPc3 human PDAC cells resulted in a marked reduction in IL-1α expression level (FIG. 4). To further investigate the contribution of the Sin3B complex in IL-1α production, BxPc3 cells were treated with trichostatin A (TSA), an inhibitor of histone deacetylases. As expected, HDAC inhibition led to the specific and dose-dependent down-regulation of IL-1α expression (FIG. 5). IL-6 expression was not significantly affected by the TSA treatment, further indicating that Sin3B is not a direct regulator of IL-6 expression (FIG. 5). Therefore, these results indicate that Sin3B and IL-1α levels are positively and functionally correlated in human pancreatic cells. Collectively, these results demonstrate that inactivation of the chromatin associated Sin3B protein impairs the occurrence of oncogene-induced senescence in a model of pancreatic ductal adenocarcinoma (PDAC), which is likely to result from a cell autonomous defect in Kras-driven production of the pro-inflammatory IL-1α cytokine. Based on the analysis herein, Sin3B-associated HDAC activity is critical in promoting KRas-driven inflammatory response, and in easing pancreatic regeneration after pancreatitis, and for this reason, may be an ideal therapeutic target.

What is claimed is:

1. A method for preventing, attenuating or inhibiting pro-oncogenic inflammation comprising administering to a subject in need thereof an effective amount of a Sin3B Complex inhibitor thereby preventing, attenuating or inhibiting pro-oncogenic inflammation.

2. The method of claim 1, wherein the subject has an activating mutation of a Kras oncogene.

3. The method of claim 2, wherein the activating mutation of the Kras oncogene is Kras^{G12D}.

4. The method of claim 1, wherein the subject is suspected of having or is at risk of having pancreatic cancer.

5. The method of claim 1, wherein the protein of the Sin3B Complex is Sin3B, HDAC1, HDAC2, KDM5A, MeCP2, SMRT, Pf1, MrgX, EMSY or GATAD1.

6. The method of claim 1, wherein the Sin3B Complex inhibitor inhibits the expression Sin3B.

7. The method of claim 1, wherein the Sin3B Complex inhibitor is a Sin3B scaffold inhibitor.

8. A method for preventing, attenuating or inhibiting initiation or progression of cancer comprising administering to a subject in need thereof an effective amount of a Sin3B Complex inhibitor thereby preventing, attenuating or inhibiting initiation or progression of cancer in the subject.

9. The method of claim 8, wherein the subject has an activating mutation of a Kras oncogene.

10. The method of claim 9, wherein the activating mutation of the Kras oncogene is Kras^{G12D}.

11. The method of claim 8, wherein the subject is suspected of having or is at risk of having pancreatic cancer.

12. The method of claim 8, wherein the protein of the Sin3B Complex is Sin3B, HDAC1, HDAC2, KDM5A, MeCP2, SMRT, Pf1, MrgX, EMSY or GATAD1.

13. The method of claim 8, wherein the Sin3B Complex inhibitor inhibits the expression Sin3B.

14. The method of claim 8, wherein the Sin3B Complex inhibitor is a Sin3B scaffold inhibitor.
